(19) United States
(12) Patent Application Publication
(10) Pub. No.: US 2022/0415448 A1
(43) Pub. Date: Dec. 29, 2022

(54) METHODS FOR FORECASTING CLINICAL COURSE OF DIFFUSE LARGE B-CELL LYMPHOMA USING RNA-BASED BIOMARKERS AND MACHINE LEARNING ALGORITHMS

(71) Applicant: Genomic Testing Cooperative, LCA, Irvine, CA (US)

(72) Inventors: Maher Albitar, Valley Center, CA (US); Hong Zhang, Savannah, GA (US)

(73) Assignee: Genomic Testing Cooperative, LCA, Irvine, CA (US)

(21) Appl. No.: 17/850,756

(22) Filed: Jun. 27, 2022

Related U.S. Application Data
(60) Provisional application No. 63/215,877, filed on Jun. 28, 2021.

(51) Int. Cl. G16B 40/20; C12Q 1/6886; G16B 20/20; G16B 30/10; G16H 50/50

(52) U.S. Cl. CPC G16B 40/20; C12Q 1/6886; G16B 20/20; G16B 30/10; G16H 50/50; C12Q 1/6869

(57) ABSTRACT

A novel classification strategy is described for forecasting clinical outcomes of Diffuse Large B-cell Lymphoma using targeted RNA sequencing combined with machine learning algorithms. The novel method classifies subjects with DLBCL into subgroups based on the clinical course of their disease and expected survival, rather than on Cell of Origin. To focus on survival, the methods first deploy machine learning and divide the subjects into subgroups based on their overall survival. A modified Bayesian classifier is then used to select genes that can forecast various survival groups, followed by validation of these biomarkers using an independent set of clinical cases. This novel approach for stratifying subjects with DLBCL based on the clinical outcome of rituximab, cyclophosphamide, doxorubicin, vincristine, and prednisone (R-CHOP) chemotherapy can be used to select high responders and low responders to R-CHOP. Low responders may be offered additional or alternative therapies to improve their survival.

### CROSS REFERENCE TO RELATED APPLICATION

This application claims the benefit of U.S. Provisional Application No. 63/215,877, filed 28 Jun. 2021, the contents of which is hereby incorporated by reference in its entirety.

### FIELD OF THE INVENTION

The field of the invention is using of machine learning algorithms for processing patient data. Specifically, the invention describes novel methods for creating a Bayesian classifier for forecasting a clinical course of diffuse large B-cell lymphoma (DLBCL) and training thereof on a training set of subjects with known survival and a known set of RNA-based biomarkers.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Diffuse large B-cell lymphoma is the most common subtype of lymphoma. However, this disease is heterogeneous, i.e., its outcome and course may vary significantly between patients (Sehn, L. H. & Salles, G. N. Diffuse large B-cell lymphoma. Engl. J. Med. 384, 842-858, 2021). Approximately 50% of patients with DLBCL can be cured with a known treatment such as rituximab, cyclophosphamide, doxorubicin, vincristine, and prednisone (R-CHOP) chemotherapy treatment. Multiple new combinations of therapeutic strategies, including additional chemotherapy agents and stem cell therapy, are being tested as additional or alternative therapies to improve survival, especially in subjects who may not respond to the known therapy (Nowakowski, G. S. & Czuczman, M. S. ABC, GCB, and Double-Hit Diffuse Large B-Cell Lymphoma: Does Subtype Make a Difference in Therapy Selection? Am. Soc. Clin. Oncol. Educ. Book. e449-57, 2015). Considering the heterogeneity of DLBCL, a single therapeutic approach is unlikely to work with all subjects with DLBCL. Therefore, multiple approaches have been used to subclassify DLBCL into various subgroups based on biological characteristics. The earliest subclassification was based on expression profiling using microarrays (Schmitz, R. et al. Genetics and pathogenesis of diffuse large B-cell lymphoma. N Engl. J. Med. 378, 1396-1407, 2018; Alizadeh, A. A. et al. Distinct types of diffuse large B-cell lymphoma identified by gene expression profiling. Nature. 403, 503-11, 2000). This classification divides DLBCL into two major groups, namely geminal center B-cell-like (GCB) and activated B-cell-like (ABC) DLBCL, based on the cell of origin (COO). In this classification, 15% of DLBCL cases were classified into the ether group. Based on a subsequent refining of this classification, the GenClass algorithm was developed. In this algorithm, genetic abnormalities are divided into four groups: MYD88 and CD79B mutations (MCD), BCL6 fusions and NOTCH2 mutations (BN2), NOTCH1 mutations (N1), and EZH2 mutations and BCL2 translocations (EZB); nevertheless, this algorithm can classify only 54% of DLBCL cases. To cover more cases, this algorithm was later extended as the LymphGen algorithm. Which divides genetic abnormalities into seven groups: MCD, N1, and B2N, as in the GenClass algorithm; MYC-negative and MYC-positive EZB; TP53 abnormality (A53) and mutations in TET2, P2RY8, or GSK1 (ST2).

Using mutation profiling and chromosomal structural abnormalities (chromosomal gains and losses), Chupy et al. classified DLBCL into five subgroups (Chapuy, B. et al. Molecular subtypes of diffuse large B cell lymphoma are associated with distinct pathogenic mechanisms and outcomes. Nat. Med. 24, 679-690, 2018). Recent FISH tests (double or triple hit) demonstrated that the rearrangement of MYC when co-present with BCL2, BCL6, or both leads to a significantly more aggressive DLBCL, making R-CHOP ineffective (Rosenthal, A. & Younes, A. High grade B-cell lymphoma with rearrangements of MYC and BCL2 and/or BCL6: Double hit and triple hit lymphomas and double expressing lymphoma. Blood. Rev. 31, 37-42, 2017; Rosenwald, A. et al. Prognostic significance of MYC rearrangement and translocation partner in diffuse large B-cell lymphoma: A study by the Lunenburg Lymphoma Biomarker Consortium. J. Clin. Oncol. 37, 3359-3368, 2019).

While existing strategies for the subclassification of DLBCLs can distinguish biologically distinct subgroups of DLBCLs, they cannot effectively predict the overall survival or progression-free survival and their distinction performance is not satisfactory. Furthermore, the clinical implementation of these classifications in routine laboratory testing is complicated by the need for performing whole exome sequencing.

Thus, even though various methods of subclassification of DLBCLs are known in the art, all or almost all of them suffer from significant drawbacks. Therefore, there remains a need for methods for classifying subjects with DLBCL into subgroups with reliably forecasted course of clinical progression of the disease and a well forecasted response to the known therapy. Low responders to the known therapy in this case may benefit from additional or alternative therapies so as to improve their overall survival.

### SUMMARY OF THE INVENTION

The inventive subject matter is directed to various compositions and methods of forecasting a clinical course of a disease for a subject with a heterogeneous disease, in particular DLBCL.

In one aspect of the inventive subject matter, the inventors contemplate a method for forecasting clinical course of a subject with a heterogeneous disease comprising the steps of providing a mathematical algorithm by classifying the subject into one of several predetermined survival groups based on response to a known therapy, obtaining the subset of individual RNA-based biomarkers for the subject, and forecasting clinical course for the subject using the subset of individual RNA-based biomarkers obtained from the subject.

In one example, the mathematical algorithm such as a Bayesian classifier, is trained using machine learning by analyzing a plurality of RNA-based biomarkers from a training set of subjects with the same heterogenous disease treated by the known therapy, each subject is characterized by their respective known plurality of individual RNA-based biomarkers and known survival time.

In another aspect of the invention, the mathematical algorithm is further trained to divide all subjects from the training set of subjects into predetermined survival groups based on survival time. In another yet aspect of the invention, the mathematical algorithm is further trained to define a subset of RNA-based biomarkers corresponding to dividing of the subjects into these groups.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a graph showing smoothing of the Bayesian prediction score to facilitate a comparison between individual RNA-based biomarkers.

FIGS. 2A and 2B show survival after respective step 1 (dividing into a first group and a second group) and step 2 (subdividing into a third, fourth, fifth, and sixth group) for forecasting of subject survival using supervised machine learning without biomarkers for the training set of subjects.

FIGS. 3A and 3B show respective actual overall survival and progression-free survival as predicted by the selected biomarkers in the training set of subjects.

FIGS. 4A and 4B shows respective predicted survival for the first and second group and for the third, fourth, fifth, and sixth groups for validation of the mathematical algorithm.

FIGS. 5A and 5B show a correlation between survival groups and two cell of origin classifications.

FIG. 6 shows several panels for assessment of a TP53 mutation as a predictor of survival.

FIGS. 7A and 7B show respective level of MYC mRNA overexpression for different survival groups and corresponding survival curves.

FIG. 8 shows IRF4 overexpression for different survival groups.

### DETAILED DESCRIPTION

The inventors have discovered various compositions and methods of forecasting clinical course and survival for subjects suffering from a heterogenous disease. For the purposes of this description, a heterogeneous disease is defined as a group of biologically diverse conditions affecting same cells or tissues and causing same or similar symptoms in a variety of subjects.

The inventors have contemplated a subject classification approach away from those based on cell origin as practiced by others. The inventors have rationalized that chromosomal structural analysis and mutation profiling eventually lead to changes in RNA profiling and activation or suppression of various pathways through relative RNA changes; thus, the RNA-based classification of DLBCL is more practical. RNA quantification may be conducted using a variety of known techniques. At the same time, a next-generation sequencing (NGS) technique has numerous advantages over other quantification methods based on microarrays and hybridization. RNA quantification by NGS is more specific and reproducible and can be performed reliably on formalin-fixed paraffin-embedded (FFPE) tissue. Furthermore, targeted RNA sequencing has the potential to be used in clinical testing because it is easier to manage and more cost effective as a routine clinical test than traditional methods.

The inventors have developed a DLBCL classification strategy for forecasting clinical outcomes using targeted RNA sequencing combined with machine learning algorithms. The novel methods classify subjects with DLBCL into subgroups based on the clinical course of their disease. To focus on survival, the methods first deploy machine learning and divide the subjects into subgroups based on their overall survival. A modified Bayesian classifier is then used to select genes that can forecast various survival groups, followed by validation of these biomarkers using an independent set of cases.

DLBCL is a heterogeneous disease with complex biological variations in the form of gene mutations, chromosomal structural abnormalities, chromosomal translocations, and microenvironment changes. Subclassification of DLBCL must account for changes in all these driving biological determinants. In principle, all these biological determinants lead to changes in the RNA levels of various genes in the tumor and microenvironment. Existing methods for the evaluation of the RNA expression and measurements of the RNA levels are highly reliable. In particular, NGS counts the number RNA molecules without significant influence of hybridization or amplification artifacts. Furthermore, targeted RNA sequencing and targeted transcriptome have a high dynamic range and can determine the biologically relevant genes and reduce the bias in sequencing of the highly expressed genes effectively. Therefore, targeted RNA expression profiling by NGS can effectively subclassify DLBCLs by encompassing all biological determinants of the clinical behavior and outcome.

However, the subclassification of a disease must reflect its clinical behavior. This is complicated by the fact that clinical behavior may be influenced by the therapy selected. The current known therapy for DLBCL is R-CHOP chemotherapy. To improve survival, subjects should be classified based on the type of response or lack of to this standard therapy. This may allow to forecast the biomarkers that determine the type of response and target the biological pathways driving these biomarkers. This approach might reduce overfitting in the process of selecting biomarkers that forecast various types of responses. In other words, instead of biomarkers forecasting survival, it might be more relevant clinically to let survival forecast biomarkers.

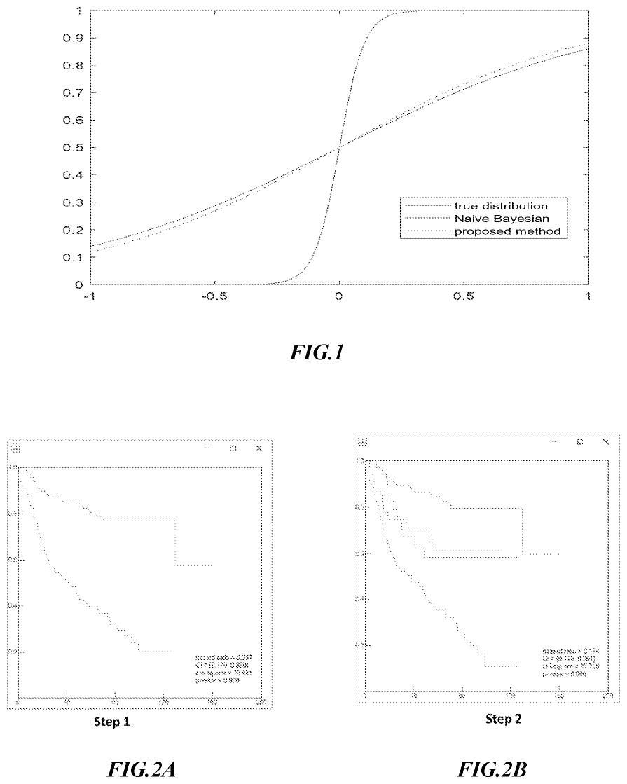

A novel forecasting method for DLBCL is described herein based on dividing a known set of subjects (referred to as a training set of subjects) into two or more groups based on survival time, rather than based on biological similarities as was done before. This approach may be used for forecasting the survival of censored subjects using machine learning. The entire training set of subjects with DLBCL is first divided into a first group of high responders and a second group of low responders. The hazard ratio was 0.237 (confidence interval: 0.170-0.330), and P-value <0.00001. The first group L of high responders is characterized by a survival time greater than the average survival time for the entire training set—see FIG. 2A. Subjects with known survival time lesser than the average for the entire set are classified into a second group S of low responders. In this description, L stands for LONG and S stands for SHORT in reference to survival time.

In a tree model, the L group of high responders is further subdivided into a third group LL and a fourth group LS, wherein the LL group is selected with survival time greater than average for the first L group. Correspondingly, the LS group is selected to include subjects from the first L group with survival time lesser than the average survival time for the first group.

Subsequently, the same sub-selection is made for the second S group of low responders, resulting in formation of the fifth SL group with survival time greater than average for the second group and the sixth SS group with survival time below the average survival for the second S group of low responders. The hazard ratio for this model was 0.174 (confidence interval: 0.120-0.251), and P-value <0.0001, see FIG. 2B.

Identification of RNA-based biomarkers is then performed following the formation of subject groups as described above. A large number of RNA-based biomarkers may be initially selected for subsequent refinement. In exemplary embodiments, the number of initial individual biomarkers is at least 500, at least 700, at least 900, at least 1000, at least 1200, at least 1400 or more. In one example described herein, the training set of subjects with known survival time and a known set of 1408 biomarkers was used to train the mathematical algorithm using machine learning. The set of individual biomarkers was generated from sequencing 1408 genes in forecasting these survival groups using naïve Bayesian statistics. Prediction using naïve Bayesian typically shows steep prediction distributions, making it difficult to compare values. Thus, the methods of the invention include a step of smoothing these distributions to facilitate a comparison between each individual biomarker, as illustrated in FIG. 1. To avoid overfitting, the training set was randomly divided into 12 different groups. Selected biomarkers were cross-validated among the 12 subgroups. This approach allowed selecting a smaller subset of ranked biomarkers, which correspond to selection of the first group of high responders and a second group of low responders. It is preferred to include between 20 and 100 individual biomarkers in the subset of ranked biomarkers. In various examples of the methods, as many as between 20 and 100; 30 and 90, 40 and 80, 50 and 70, or 60 individual biomarkers are selected as ranked and allowing to differentiate between the first group of high responders and the second group of low responders to the known therapy.

In view of the foregoing, a method for treating a subject with a heterogeneous disease, such as diffuse large B-cell lymphoma, is provided. The method may include providing a mathematical algorithm for forecasting clinical course of the subject with the heterogeneous disease by classifying the subject into one of several predetermined survival groups based on response to a known therapy. The mathematical algorithm may be trained using machine learning by analyzing a plurality of RNA-based biomarkers from a training set of subjects with the same heterogenous disease treated by the known therapy, each subject may be characterized by their respective known plurality of individual RNA-based biomarkers and known survival time. The mathematical algorithm may be further trained to divide all subjects from the training set of subjects into predetermined survival groups based on survival time. The mathematical algorithm is further trained to define a subset of RNA-based biomarkers corresponding thereto. The method may further includes obtaining the subset of individual RNA-based biomarkers defined in step (a) for the subject. The method may further include forecasting clinical course for the subject using the subset of individual RNA-based biomarkers obtained from the subject. The method may further include treating the subject forecasted in step (c) with the known therapy.

In some embodiments, a method for identifying one or more individual RNA-based biomarkers for forecasting clinical course of the subject with the heterogeneous disease is provided. The method includes providing a training set of subjects with the heterogenous disease with known plurality of individual RNA-based biomarkers and known survival time. The method may further include based on survival time, dividing all subjects from the training set into a first group of high responders and a second group of low responders. The method may further include using machine learning, identifying a first subset of one or more individual RNA-based biomarkers from a plurality of individual RNA-based biomarkers, wherein the first subset of one or more individual RNA-based biomarkers is identified as correlating to dividing the subjects into the first group and the second group.

In other embodiments, a method for treating a subject with diffuse large B-cell lymphoma is provided. The method includes a step of using a Bayesian classifier to define the subject as a high responder or a low responder to chemotherapy using one or more of individual RNA-based biomarkers selected from a group consisting of PPP2R1B, GOLGA5, LINGO2, HMGA1, SIN3A, ARID1A, BCL7A, CDK5RAP2, MAGED1, CREB3L1, AMER1, DLL1, GSTT1, GPR34, DNM2, CCNB1IP1, MUTYH, RET, CDH1, POFUT1, XRCC6, KIT, RALGDS, SS18, CD22, BRCA2, HDAC3, LHX4, FAM19A2, PRG2, PRCC, TBL1XR1, HIF1A, EDIL3, ROS1, DKK4, CDC25A, WNT7B, MYBL1, MLLT10, SLCO1B3, TACC2, CANT1, NCAM1, FGF3, FGF19, PPP3R2, CRADD, ETV6, SPP1, SDHB, FGF2, SUZ12, MB21D2, MYC, BAX, CEP57, ITGA5, ABCC3, and HECW1.

In one example shown in FIG. 2A, the ranked subset of individual biomarkers included 60 selected genes as listed here: PPP2R1B, GOLGA5, LINGO2, HMGA1, SIN3A, ARID1A, BCL7A, CDK5RAP2, MAGED1, CREB3L1, AMER1, DLL1, GSTT1, GPR34, DNM2, CCNB1IP1, MUTYH, RET, CDH1, POFUT1, XRCC6, KIT, RALGDS, SS18, CD22, BRCA2, HDAC3, LHX4, FAM19A2, PRG2, PRCC, TBL1XR1, HIF1A, EDIL3, ROS1, DKK4, CDC25A, WNT7B, MYBL1, MLLT10, SLCO1B3, TACC2, CANT1, NCAM1, FGF3, FGF19, PPP3R2, CRADD, ETV6, SPP1, SDHB, FGF2, SUZ12, MB21D2, MYC, BAX, CEP57, ITGA5, ABCC3, and HECW1.

The same approach is then done for the subdivision of the first group of high responders to the third LL group and the fourth LS group. A similar number of ranked biomarkers (60 in this example) is selected to correspond to this step—as shown in FIG. 2B. The second L group is similarly divided into the SL and SS groups with its own subset of ranked individual biomarkers.

The second set of biomarkers for subdividing the first group of high responders is listed here: DUSP22, CTNNA1, DUX2, SSX1, SSX2, CTNNB1, DCLK2, FH, DUSP9, FCGR2B, STAT5B, ESR1, CD274, TERF1, AKAP9, DGKI, HMGA1, ARNT, MAFB, PPP3CC, COL3A1, NUTM2A, CIT, MGMT, CDK6, SORT1, RCSD1, CDK5RAP2, SIN3A, RABEP1, MB21D2, KDR, SS18L1, SSBP2, SH2D5, ASXL1, AMER1, AFF1, PRKCD, 2-Sep, TPM4, FIGF, NODAL, GRM3, STAT6, GAB1, RPL22, BDNF, SNX29, MELK, ARRDC4, FGF10, MMP9, YY1AP1, HAS2, DLEC1, DEK, TLL2, BCL2L2, and ID3.

The third set of ranked biomarkers for subdividing the second group of low responders is listed here: AHI1, EPHA5, DUSP22, DUSP26, DUSP9, DUX2, MGMT, MIB1, MIPOL1, MIR1260B, MIR4321, MIR4683, MIR4758, MIR6515, MIR6752, MIR6765, BIVM-ERCC5, SSX1, SSX2, LTBP1, MAFB, TLR4, CTNNB1, ETV5, CHEK2, FUS, SS18L1, SSBP2, DGKI, CIT, TFE3, FGF19, TRIM33, CTCF, LAMA1, TBL1XR1, TOP1, RB1, OLR1, DOCK1, ARID1A, RABEP1, EP400, STK11, ETS1, MAPK1, CDC14A, LMO7, SS18, ICK, FLI1, POU5F1, RCSD1, HRAS, BACH2, CDK7, GAS5, CARS, SRSF2, and MAP3K6.

There was very little overlap among the three sets of ranked biomarkers. As shown in FIG. 2, the overall survival rates of LS and SL groups were similar. However, completely different sets of genes were used for selecting each group. This indicates that even though these two groups have similar clinical courses, they are completely biologically different. This reflects the significant heterogeneity of DLBCL.

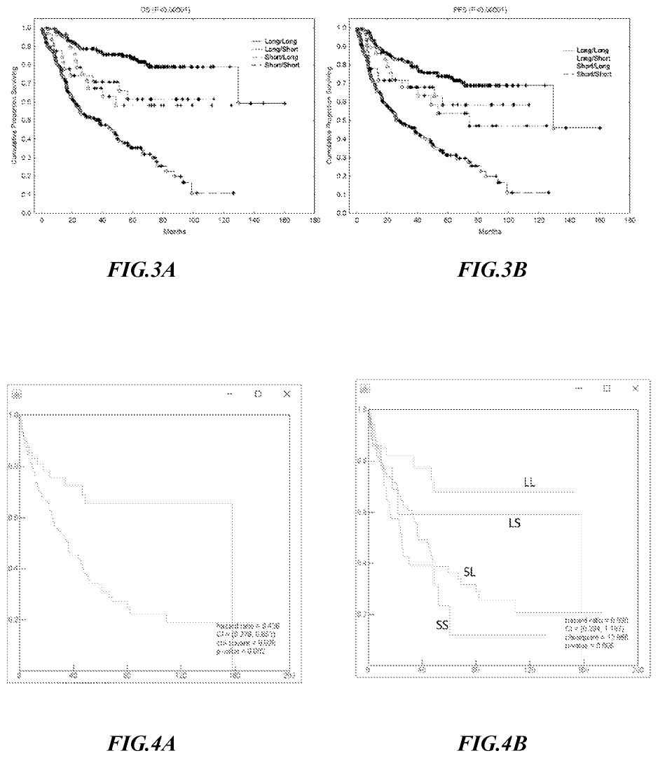

Using the selected biomarkers, we classified the subjects in the original set (379 subjects) into LL, LS, SL, and SS groups and then evaluated the survival pattern of these groups. As shown in FIG. 3A, the selected biomarkers forecasted survival as expected in the overall survival groups prior to biomarker selection. The same was true for the forecasted progression-free survival (FIG. 3B).

To further validate these biomarkers, an independent group of subjects was used, 247 subjects, in one example, with extranodal DLBCL. As shown in FIGS. 4A and 4B, these biomarkers efficiently forecasted survival in the extranodal subjects despite the shorter overall survival, with an HR of 0.26 (confidence interval: 0.278-0.653, P-value=0.002), as well as when they were divided into four groups using the three sets of biomarkers with an HR of 0.530 (confidence interval: 0.234-1.197, P=0.005) (FIG. 4). As expected, extranodal DLBCL leads to overall shorter survival and more aggressive disease.

The classification based on survival methods of the invention was then correlated with COO classification, TP53 mutation status, MYC expression, and IRF4 expression. However, in the multivariate analysis, only TP53 mutations were independent in forecasting prognosis, see Table 1 below.

TABLE 1
Multivariate survival analysis

| | | | | | | | | | Risk | Risk |
|---|---|---|---|---|---|---|---|---|---|---|
| | | | Beta | Beta | | | | | ratio | ratio |
| | | | 95% | 95% | t- | | | Risk | 95% | 95% |
| N = 379 | Beta | Standard | lower | upper | value | Wald | P | ratio | lower | upper |
| Survival | 0.58 | 0.07 | 0.43 | 0.73 | 7.79 | 60.65 | 0.00000 | 1.78 | 1.54 | 2.07 |
| classification | | | | | | | | | | |
| Age60 | 0.47 | 0.18 | 0.11 | 0.83 | 2.57 | 6.61 | 0.01017 | 1.60 | 1.12 | 2.30 |
| GCB vs ABC | −0.12 | 0.18 | −0.48 | 0.24 | −0.65 | 0.42 | 0.51873 | 0.89 | 0.62 | 1.27 |
| Survival | 0.56 | 0.07 | 0.41 | 0.70 | 7.49 | 56.16 | 0.00000 | 1.74 | 1.51 | 2.01 |
| classification | | | | | | | | | | |
| Age60 | 0.47 | 0.18 | 0.11 | 0.83 | 2.54 | 6.47 | 0.01100 | 1.60 | 1.11 | 2.29 |
| COO | 0.01 | 0.18 | −0.35 | 0.37 | 0.06 | 0.00 | 0.95425 | 1.01 | 0.70 | 1.45 |
| Classification | | | | | | | | | | |
| Mute.TP53 | 0.50 | 0.18 | 0.14 | 0.86 | 2.74 | 7.53 | 0.00608 | 1.65 | 1.15 | 2.36 |
| Survival | 0.57 | 0.07 | 0.43 | 0.72 | 7.64 | 58.35 | 0.000000 | 1.77 | 1.53 | 2.05 |
| classification | | | | | | | | | | |
| Age60 | 0.50 | 0.19 | 0.14 | 0.86 | 2.70 | 7.31 | 0.006864 | 1.65 | 1.15 | 2.37 |
| COO | 0.05 | 0.19 | −0.33 | 0.42 | 0.25 | 0.06 | 0.80395 | 1.05 | 0.72 | 1.52 |
| Classification | | | | | | | | | | |
| Mute. MYD88 | −0.39 | 0.22 | −0.82 | 0.04 | −1.78 | 3.16 | 0.075324 | 0.68 | 0.44 | 1.04 |
| Mute. CD79B | −0.22 | 0.32 | −0.84 | 0.40 | −0.69 | 0.47 | 0.492658 | 0.81 | 0.43 | 1.50 |
| Mute. TP53 | 0.46 | 0.18 | 0.10 | 0.82 | 2.50 | 6.26 | 0.012322 | 1.59 | 1.11 | 2.28 |
| Survival | 0.57 | 0.08 | 0.42 | 0.71 | 7.41 | 54.95 | 0.000000 | 1.76 | 1.52 | 2.04 |
| classification | | | | | | | | | | |
| Classification | 0.06 | 0.18 | −0.29 | 0.42 | 0.33 | 0.11 | 0.737781 | 1.06 | 0.74 | 1.52 |
| Mute. TP53 | 0.47 | 0.19 | 0.11 | 0.84 | 2.55 | 6.53 | 0.010635 | 1.61 | 1.12 | 2.31 |
| MYC U25% | 0.01 | 0.18 | −0.34 | 0.37 | 0.07 | 0.00 | 0.948052 | 1.01 | 0.71 | 1.44 |
| Survival | 0.58 | 0.08 | 0.43 | 0.73 | 7.73 | 59.71 | 0.000000 | 1.79 | 1.54 | 2.07 |
| classification | | | | | | | | | | |
| Classification | 0.05 | 0.18 | −0.31 | 0.40 | 0.27 | 0.07 | 0.790027 | 1.05 | 0.74 | 1.50 |
| Mute. TP53 | 0.50 | 0.18 | 0.14 | 0.86 | 2.70 | 7.31 | 0.006849 | 1.65 | 1.15 | 2.37 |
| MYC | 0.00 | 0.00 | 0.00 | 0.00 | −1.14 | 1.31 | 0.252632 | 1.00 | 1.00 | 1.00 |
| Survival | 0.60 | 0.08 | 0.45 | 0.75 | 7.85 | 61.65 | 0.000000 | 1.83 | 1.57 | 2.12 |
| Classification | | | | | | | | | | |
| Age60 | 0.46 | 0.18 | 0.10 | 0.82 | 2.49 | 6.21 | 0.012719 | 1.58 | 1.10 | 2.27 |
| COO | 0.16 | 0.21 | −0.26 | 0.57 | 0.73 | 0.54 | 0.463977 | 1.17 | 0.77 | 1.77 |
| classification | | | | | | | | | | |
| Mute. TP53 | 0.51 | 0.19 | 0.15 | 0.88 | 2.76 | 7.61 | 0.00582 | 1.67 | 1.16 | 2.41 |
| MYC mRNA | 0.00 | 0.00 | 0.00 | 0.00 | −1.11 | 1.24 | 0.265004 | 1.00 | 1.00 | 1.00 |
| IRF4 mRNA | 0.00 | 0.00 | 0.00 | 0.00 | −2.02 | 4.08 | 0.0433 | 1.00 | 1.00 | 1.00 |

### Correlation with Cell of Origin (COO) Classification

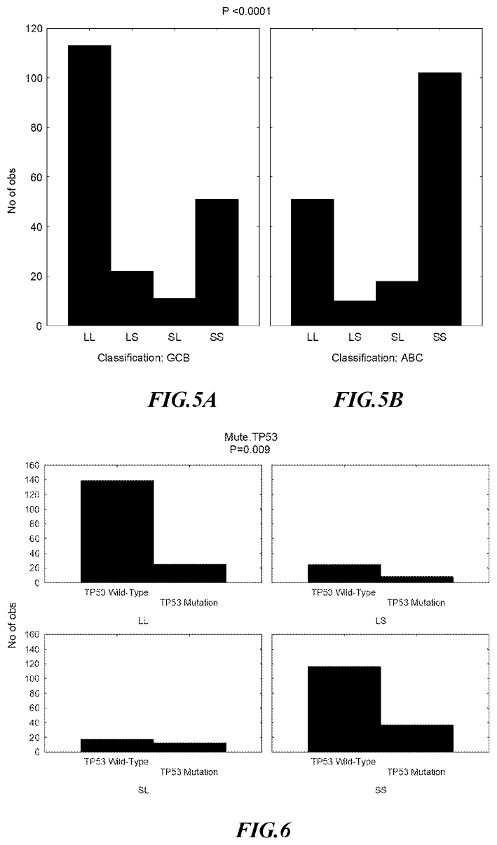

The training set of 379 subjects was also classified as cells of origin. The prevalence of ABC and GCB mutations in our survival groups was evaluated. The majority of the GCB cases had a good prognosis (LL and LS; P<0.0001), see FIG. 5. Furthermore, although the LS and SL groups showed similar overall survival, there were significantly more GCB cases in the LS group than in the SL group (P=0.016). This also confirms that, despite having similar outcomes, the LS and SL groups are biologically different.

In the multivariate model incorporating the survival classification with COO and the age of subjects (younger vs. older than 60 years), survival classification and age grouping were independent predictors of survival, but COO was no longer a predictor of survival (Table 1).

### Correlation with TP53 Mutation

Of the 379 DLBCL subjects, 82 (22%) had TP53 mutations. As expected, subjects with TP53 had significantly shorter survival rates (p=0.0019). There were relatively more TP53 mutations in the short survival groups (P=0.009), FIG. 6. More importantly, in the multivariate model incorporating TP53 mutation with survival classification, age, and COO, TP53 mutations remained strong independent predictors of survival (Table 1).

### Correlation with MYD88 and CD79B Mutations

Subjects with MYD88 mutations were more common in the S group (P=0.001) with aggressive DLBCL. However, there was no significant difference in the distribution of subjects with CD79B mutations among the various survival groups (P=0.49). In the multivariate model incorporating mutations in TP53, CD79B, and MYD88 along with COO, age, and survival classification, mutations in CD79B and MYD88 were no longer predictors of survival, whereas TP53 mutation remained a predictor of survival (Table 1).

### Correlation with MYC Overexpression

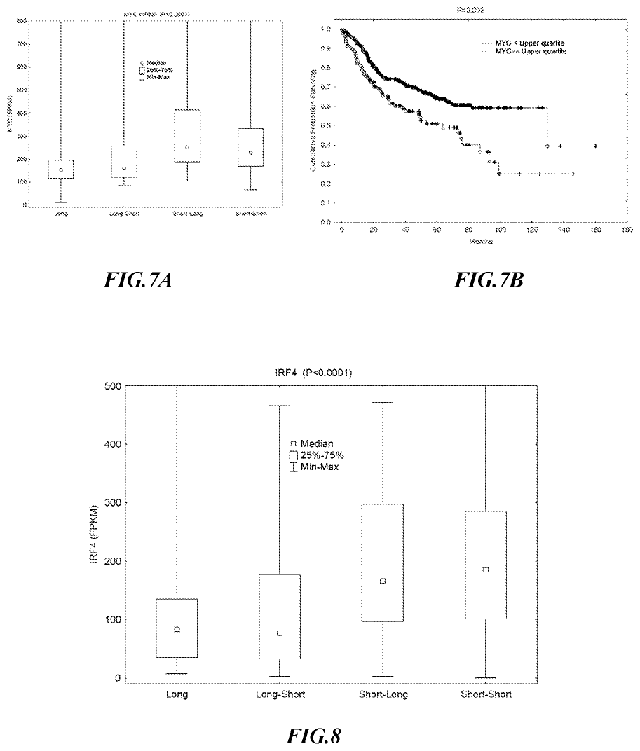

MYC expression was significantly higher in the S groups (P<0.0001). Higher levels of MYC mRNA were detected in the SL group than in the LS group (P<P-0.001), although the two groups showed similar survival (FIG. 7). Short survival was associated with high MYC expression when used as a continuous variable (P=0.0019) or when subjects were grouped as low vs. high based on the upper quartile (P=0.0021), FIG. 7. However, in the multivariate model, MYC expression was not an independent predictor of survival, irrespective of whether it was used as a continuous and categorical (low vs. high) variable (Table 1).

### Correlation with IRF4 Overexpression

IRF4 gene translocation is typically associated with overexpression.^{12,14} Recent studies have shown that DLBCL with IRF4 translocation is less damaging. IRF4 RNA overexpression was investigated for correlation with the survival groups, as forecasted in the model. Significant overexpression of IRF4 mRNA was observed in the S group of subjects (FIG. 8). As well as lower levels of MYC, the LS group had significantly lower levels of IRF4 mRNA than the SL group (P=0.02), although there was no difference in survival between these two groups. In the multivariate model incorporating the survival groups, among COO, TP53, and IRF4 mRNA as continuous variables, IRF4 mRNA level and TP53 mutation were independent negative predictors of survival (Table 1).

These findings confirm that the subclassification of subjects using survival is a reliable approach to define biologically different subjects with DLBCL. In fact, although the LS and SL groups had similar survival, they had significantly different MYC and IRF4 levels. This supports the assumption that it is unrealistic to assume that one biomarker can define specific clinical behavior and that significant overlap between biomarkers exists in driving the biology of DLBCL.

As the objective of this classification is to forecast clinical course progression of the DLBCL subjects, it is important to accurately predict who will respond well to a known therapy (high responders) and who will not (low responders). The known therapy in this case is a chemotherapy using a predetermined combination of rituximab, cyclophosphamide, doxorubicin hydrochloride, vincristine sulfate, and prednisone, referred to as R-CHOP. Low responders, and especially the subjects in the SS group may be referred to additional or alternative treatments. Examples of such additional or alternative treatments include additional chemotherapy agents such as etoposide. Further therapies include such examples as stem transplant therapy, and specifically an autologous stem transplant therapy. The methods of the invention may be further used to select appropriate candidates for clinical trials of yet to be developed therapies for treating DLBCL. It may be easier to find a new successful therapeutic approach when subjects with similar biology and clinical courses are treated in clinical trials with new therapeutic regimens.

This subclassification of DLBCL subjects can be automated through a software with RNA sequencing data as an input for individual subjects. Such software is configured to run on a computer system featuring a processor, a readable memory, and other components facilitating operation of the computer system to first train the mathematical algorithm using a training set of subjects and then use thereof for forecasting a clinical course for individual subjects.

### EXAMPLES

### Subjects

RNA sequencing using a targeted panel was performed on samples from 379 subjects with de novo DLBCL and 247 subjects with extranodal DLBCL. A total of 379 patents were used to establish the prognostic model, and 247 subjects were used for validation. All subjects were treated with a known therapy of R-CHOP chemotherapy. These samples were collected from 22 medical centers organized for retrospective studies as part of the DLBCL Consortium Program. This study was approved by the institutional review board of each participating medical center and was conducted in accordance with the Declaration of Helsinki. Subjects with transformed DLBCL, primary mediastinal large B-cell lymphoma, primary central nervous system DLBCL, or primary cutaneous DLBCL were excluded.

### RNA Library Construction and Sequencing

The Agencourt FormaPure Total 96-Prep Kit was used to extract DNA and RNA from the same FFPE tissue lysates using an automated KingFisher Flex following the protocols recommended by the manufacturers. Samples were selectively enriched for 1408 cancer-associated genes using reagents provided in the Illumina® TruSight® RNA Pan-Cancer Panel. cDNA was generated from the cleaved RNA fragments using random primers during the first and second strand synthesis. Sequencing adapters were ligated to the resulting double-stranded cDNA fragments. The coding regions of the expressed genes were captured from this library using sequence-specific probes to create the final library. Sequencing was performed using an Illumina NextSeq 550 system platform. Ten million reads per sample in a single run were required, and the read length was 2×150 bp. The sequencing depth was 10×-1739× with a median of 41×. An expression profile was generated from the sequencing coverage profile of each individual sample using Cufflinks. Expression levels were measured as fragments per kilobase of transcript per million.

### Machine Learning Methods for Survival Analysis

A machine learning method was used to estimate the survival time of a censored subject with no know the survival time, using the Kaplan-Meier curve.

Theorem. Let S(t) be the survival function and f (t) be the probability density function of survival. For a censored case at time t₀, the conditional expected survival time is

$${t}_{0} + \frac{1}{S ( {t}_{0} )} {\int }_{{t}_{0}}^{∞} S ( t ) dt .$$

Proof. Given the censored time t₀, the conditional density function is

$$\frac{f ( t )}{S ( {t}_{0} )} , t \geq {t}_{0} ,$$

and the expectation is

$$\begin{matrix}{\int }_{{t}_{0}}^{∞} t \frac{f ( t )}{S ( {t}_{0} )} dt = \frac{1}{S ( {t}_{0} )} {\int }_{{t}_{0}}^{∞} td [ - S ( t ) ] \\ = - \frac{1}{S ( {t}_{0} )} tS ( t ) {\vert}_{{t}_{0}}^{∞} + \frac{t}{S ( {t}_{0} )} {\int }_{{t}_{0}}^{∞} S ( t ) dt \\ = {t}_{0} + \frac{1}{S ( {t}_{0} )} {\int }_{{t}_{0}}^{∞} S ( t ) dt .\end{matrix}$$

However, the conditional expectation given in the theorem may not be an appropriate label for the machine learning algorithm. The formula does not consider the confidence of the estimation; it will always return a value greater than the mean survival and have a bias toward the long survival class. To address this problem, the survival is estimated as follows:

$$survival = \{ \begin{matrix}mean , if {t}_{0} \leq \frac{mean}{2} \\ {t}_{0} + \frac{1}{S ( {t}_{0} )} {\int }_{{t}_{0}}^{∞} S ( t ) dt , if {t}_{0} > \frac{mean}{2}\end{matrix} .$$

To select biomarkers for the prediction of survival groups, a naïve Bayesian classifier is used. However, Bayesian classifiers suffer from severe numerical underflow problems when the dimension of the data is high. Even with careful scaling, all but the dominant feature is still likely to underflow. To solve this problem, a generalized naïve Bayesian classifier is developed by applying a geometric mean to the likelihood product. This proves that this approach eliminates the underflow problem, and the geometric mean is the only function satisfying these conditions.

The naïve Bayesian classifier is an effective machine learning algorithm. It is based on Bayes' theorem and the assumption that all attributes are conditionally independent. Let (x₁, x₂, . . . , x_{d}) be the input attribute vector and (C₁, C₂, . . . , Cₖ) be the classes. According to Bayes Theorem,

$$P ( {C}_{j} \vert {x}_{1} , {x}_{2} , … , {x}_{d} ) = \frac{P ( {C}_{j} ) P ( {x}_{1} , {x}_{2} , … , {x}_{d} \vert {C}_{j} )}{{\sum }_{i = 1}^{K} P ( {C}_{i} ) P ( {x}_{1} , {x}_{2} , … , {x}_{d} \vert {C}_{i} )} .$$

With the assumption of conditional independence,

P(x₁,x₂, . . . ,x_{d}|Cⱼ)=P(x₁|Cⱼ)P(x₂|Cⱼ) . . . P(x_{d}|Cⱼ).

The probabilities P(xᵢ|Cⱼ) can be estimated from the training set data. However, when the dimension d is large, the products of the probabilities (likelihood) become extremely small, causing underflows. If each probability value has an average of ½, the likelihood will have a mean

$$E [ P ( {x}_{1} \vert {C}_{j} ) P ( {x}_{2} \vert {C}_{j} ) … P ( {x}_{d} \vert {C}_{j} ) ] = \frac{1}{{2}^{d}} ,$$

which approaches 0 quickly when d is large.

One typical method to avoid numerical underflow is to scale all the values using the largest probability product during the computations. However, this method often produces one value that dominates the probability products. As a result, one class will have the forecasted probability of 1.0 while all other classes will have a prediction probability of 0.0. This effect is disadvantageous for most applications because it is an artifact of the naïve Bayesian assumption and usually does not reflect the real probability.

The inventors have developed a novel generalization to the standard naïve Bayesian algorithm to address the underflow problem. Let h(x) be a positive increasing function. Applying the function to the likelihood produces a new probability estimate:

P(x₁,x₂, . . . ,x_{d}|Cⱼ)=h[P(x₁|Cⱼ)P(x₂|Cⱼ) . . . P(x_{d}|Cⱼ)].

In particular, the function

h(x,d)=x^{1/d},

is used, which increases monotonically with d and prevents underflow for any dimension d.

Lemma. Let x be a uniform random value over the interval [0,1]; the expected value of x h(x,d)=x^{1/d} for a constant d is

$$\frac{1}{( 1 + 1 / d )} .$$

Proof. Because x is uniform, the expected value of x^{1/d} is

$${\int }_{0}^{1} {x}^{1 / d} dx = \frac{{x}^{1 + 1 / d}}{1 + 1 / d} {\vert}_{0}^{1} = \frac{1}{( 1 + 1 / d )} .$$

Theorem. Assume that the probabilities in the likelihood are independent, uniformly distributed random variables. Then, the expected value of the likelihood is

$$E [ {( P ( {x}_{1} \vert {C}_{j} ) P ( {x}_{2} \vert {C}_{j} ) … P ( {x}_{d} \vert {C}_{j} ) )}^{1 / d} ] = \frac{1}{{( 1 + 1 / d )}^{d}} .$$

Proof. By the previous lemma and the independence of the random variables,

$$E [ {( P ( {x}_{1} \vert {C}_{j} ) P ( {x}_{2} \vert {C}_{j} ) … P ( {x}_{d} \vert {C}_{j} ) )}^{\frac{1}{d}} ] = E [ {( P ( {x}_{1} \vert {C}_{j} ) )}^{\frac{1}{d}} [ E [ {( P ( {x}_{2} \vert {C}_{j} ) )}^{\frac{1}{d}} ] … E [ {( P ( {x}_{d} \vert {C}_{j} ) )}^{\frac{1}{d}} ] = \frac{1}{{( 1 + 1 / d )}^{d}} .$$

The limit of the expected value is

$${lim}_{d \to ∞} \frac{1}{{( 1 + 1 / d )}^{d}} = 1 / e .$$

Therefore, as the dimension increases, the likelihood will never approach 0 uniformly.

Applying the function h to the likelihood does not change the relative order of the probability estimates of the classes. However, the probabilities will have more reasonable values than 0 and 1.

Importantly, the function h(x, d)=x^{1/d} is unique under certain conditions.

Lemma. Let f(x) be a positive continuous function of positive real numbers. If f is multiplicative, f(xy)=f(x)f(y), then f(x)=x^{a} for some constant a.

In the case of the functional transform on the likelihood, the assumption of the multiplicative property on the function h is a natural extension of the naïve Bayesian assumption.

By requiring that the likelihood approaches a non-zero limit as d approaches infinity, the function has the form h(x,d)=x^{c/d} for a constant c.

Theorem. If h is multiplicative and

$${lim}_{d \to ∞} E [ h ( P ( {x}_{1} \vert {C}_{j} ) P ( {x}_{2} \vert {C}_{j} ) … P ( {x}_{d} \vert {C}_{j} ) ) ] = L > 0 ,$$

then h(x,d)=x^{a(d)}, where

$$a ( d ) = c ( \frac{1}{d} ) + O ( \frac{1}{{d}^{2}} ) , c > 0.$$

Proof. The previous lemma shows that

h(x,d)=x^{a(d)}.

Similar to the previous proof, the expectation is

$$E [ h ( P ( {x}_{1} \vert {C}_{j} ) P ( {x}_{2} \vert {C}_{j} ) … P ( {x}_{d} \vert {C}_{j} ) ) ] = E [ {( P ( {x}_{1} \vert {C}_{j} ) P ( {x}_{2} \vert {C}_{j} ) … P ( {x}_{d} \vert {C}_{j} ) )}^{a ( d )} ] = E [ {( P ( {x}_{1} \vert {C}_{j} ) )}^{a ( d )} ] E [ {( P ( {x}_{2} \vert {C}_{j} ) )}^{a ( d )} ] … E [ {( P ( {x}_{d} \vert {C}_{j} ) )}^{a ( d )} ] = \frac{1}{{( 1 + a ( d ) )}^{d}} .$$

By the assumption, there is the following:

$${lim}_{d \to ∞} \frac{1}{{( 1 + a ( d ) )}^{d}} = L > 0 .$$

Letting t=1/d and f(t)=a(1/t)=a(d), then

$${lim}_{d \to ∞} \frac{1}{{( 1 + a ( d ) )}^{d}} = {lim}_{t \to 0 +} \frac{1}{{( 1 + f ( t ) )}^{\frac{1}{t}}} = {lim}_{t \to 0 +} {e}^{\frac{- ln ( 1 + f ( t ) )}{t}} .$$

Furthermore, f(0+)=0 and

$${lim}_{t \to 0} {e}^{\frac{- ln ( 1 + f ( t ) )}{t}} = {lim}_{t \to 0} {e}^{\frac{- {f}^{′} ( t )}{1 + f ( t )}} = {lim}_{t \to 0} {e}^{- {f}^{′} ( t )} = {e}^{- c} = L .$$

Therefore,

$$f ( t ) = ct + O ( {t}^{2} ) ,$$

$$a ( d ) = c ( \frac{1}{d} ) + O ( \frac{1}{{d}^{2}} ) , c > 0 .$$

When the dimension d is high, the independence assumption of the naïve Bayesian classifier is unlikely to be true in most applications. Consequently, the probability estimates are unrealistic. The proposed extension as described below solves this problem.

Example. Consider a two-class problem with d-dimensional Gaussian distributions, with means of

(1,1, . . . ,1) and (−1, −1, . . . , −1) and the same covariance matrix

$$[ \begin{matrix}1 & r & … & r \\ r & 1 & … & r \\ ⋮ & ⋮ & ⋱ & ⋮ \\ r & r & … & 1\end{matrix} ] = ( 1 - r ) I + rJ ;$$

the inverse matrix is

$$\frac{1}{1 - r} ( I - \frac{r}{1 - r + rd} J ) .$$

Consider the probability estimations for the point (t, t, . . . , t). The true probability for class 1 is

$$\frac{{e}^{- 0.5 {d ( t - 1 )}^{2} ( 1 - \frac{rd}{1 - r + rd} )}}{{e}^{- 0.5 {d ( t - 1 )}^{2} ( 1 - \frac{rd}{1 - r + rd} )} + {e}^{- 05 {a ( t + 1 )}^{2} ( 1 - \frac{rd}{1 - r + rd} )}}$$

For the original naïve Bayesian classifier,

$$\frac{{e}^{- 0.5 {d ( t - 1 )}^{2}}}{{e}^{- 0.5 {d ( t - 1 )}^{2}} + {e}^{- 0.5 {d ( t + 1 )}^{2}}} ,$$

and for the proposed classifier,

$$\frac{{e}^{- 0.5 {( t - 1 )}^{2}}}{{e}^{- 0.5 {( t - 1 )}^{2}} + {e}^{- 0.5 {( t + 1 )}^{2}}} .$$

FIG. 1 shows the three probability estimates for d=10 and r=0.5. The naïve Bayesian probability estimates change steeply around the boundary owing to the independence assumption. In contrast, our proposed method closely approximates the true probabilities.

### Feature Selection

A discriminant measure for single genes was used to facilitate gene selection. This method was based on cross-validation to avoid overfitting. This measure is consistent with the generalized naïve Bayesian classifier. To fully utilize the survival data, a parameter estimation method on the means and variations was used for the generalized naïve Bayesian classifier. By modeling the relationship between survival time and classes, an improved formula for estimating the means and variances of the distributions was obtained.

A single level of gene selection and classification for this survival analysis problem is not adequate for detecting groups defined by NGS biomarkers. Thus, a hierarchical approach was developed to use multiple levels of gene selection and classification for the prediction of survival as well as the detection of biomarker-related groups. Owing to the inherent uncertainties in the survival data, it is usually not feasible to include a large number of genes in machine learning algorithms. Thus, a subset of genes relevant to the prediction task was selected.

Standard dimension reduction methods, such as principal component analysis (PCA) and recursive feature elimination, start with a system with all features included. It would be difficult to obtain effective features from noisy survival data in such a highly over-fitted and volatile system. In PCA-based methods, it is also difficult to extract an explicit gene list because the mappings would involve the entire set of genes. Following the same principle applied in the naïve Bayesian classifier, we propose a feature selection method to select and rank genes based on a discriminant measure of individual genes.

To reduce the effects of noise and avoid overfitting, a k-fold cross-validation was used to obtain a robust measure. For an individual gene, a generalized naïve Bayesian classifier was constructed on the training subset and tested on the testing subset. The complement d₁₂ of the cross-validation error rate was used as a discriminant measure for the gene.

d₁₂=1−error₁₂

The genes were ranked by d₁₂; higher values corresponded to more relevant genes for classifying the two classes.

The survival data consisted of continuous values that did not represent a class label directly; however, the magnitude of the values provide useful information on the class. We estimated the mean and variance of the distribution in the generalized naïve Bayesian classifier by weighted averages based on the relationship between survival time and class membership.

Let y be the survival time and P(Cₖ|y) be the conditional probability function connecting y and class Cₖ. Assuming that there are two classes and P(y|Cₖ),k=1,2 are Gaussian with equal variances, according to Bayes' theorem,

$$P ( {C}_{k} \vert y ) = \frac{P ( y \vert {C}_{k} ) P ( {C}_{k} )}{P ( y \vert {C}_{1} ) P ( {C}_{1} ) + P ( y \vert {C}_{2} ) P ( {C}_{2} )} = \frac{1}{1 + {e}^{a ( y - b )}} ,$$

which is a logistic function.

Given the training cases (xᵢ,yᵢ), i=1,2, . . . , n, then the likelihood function

L=−Σᵢ₌₁ⁿln[Σₖ₌₁²P(Cₖ|y)P(xᵢ|Cₖ)].

Maximizing the likelihood,

$$\frac{\partial L}{\partial {m}_{k}} = {\sum }_{i = 1}^{n} \frac{P ( {C}_{k} \vert {y}_{i} ) P ( {x}_{i} \vert {C}_{k}}{{\sum }_{k = 1}^{2} P ( {C}_{k} \vert {y}_{i} ) P ( {x}_{i} \vert {C}_{k} )} ( {x}_{i} - {m}_{k} ) = 0 .$$

The coefficients involve unknown values P(xᵢ|Cₖ). If they are set as constants, one can solve the equations and obtain an explicit formula for the means:

$${m}_{k} = {\sum }_{i = 1}^{n} \frac{P ( {C}_{k} \vert {y}_{i} ) {x}_{i}}{{\sum }_{j = 1}^{n} P ( {C}_{k} \vert {y}_{j} )} = {\sum }_{i = 1}^{n} {w}_{i} {x}_{i} ,$$

where is the weighted average of xᵢ. The weights are proportional to the class probability on yᵢ:

$${w}_{i} = \frac{P ( {C}_{k} \vert {y}_{i} )}{{\sum }_{j = 1}^{n} P ( {C}_{k} \vert {y}_{j} )} .$$

Similarly, the variances can be estimated as follows:

$${σ}_{k}^{2} = {\sum }_{i = 1}^{n} \frac{P ( {C}_{k} \vert {y}_{i} ) {( {x}_{i} - {m}_{k} )}^{2}}{{\sum }_{j = 1}^{n} P ( {C}_{k} \vert {y}_{j} )} = {\sum }_{i = 1}^{n} {{w}_{i} ( {x}_{i} - {m}_{k} )}^{2} .$$

Further aspects and considerations are described in Blood Cancer Journal (2022) 12:25 and the supplementary information thereto, the entirety of which is incorporated by reference herein.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A method for treating a subject with a heterogeneous disease, wherein the heterogeneous disease is defined as a group of biologically diverse conditions affecting same cells or tissues and causing same or similar symptoms, the method comprising:
a. providing a mathematical algorithm for forecasting clinical course of the subject with the heterogeneous disease by classifying the subject into one of several predetermined survival groups based on response to a known therapy,
wherein the mathematical algorithm is trained using machine learning by analyzing a plurality of RNA-based biomarkers from a training set of subjects with the same heterogenous disease treated by the known therapy, each subject is characterized by their respective known plurality of individual RNA-based biomarkers and known survival time, and
wherein the mathematical algorithm is further trained to divide all subjects from the training set of subjects into predetermined survival groups based on survival time, the mathematical algorithm is further trained to define a subset of RNA-based biomarkers corresponding thereto;
b. obtaining the subset of individual RNA-based biomarkers defined in step (a) for the subject;
c. forecasting clinical course for the subject using the subset of individual RNA-based biomarkers obtained from the subject; and
d. treating the subject forecasted in step (c) with the known therapy.

2. The method as in claim 1, wherein in step (a) the mathematical algorithm is further trained to divide all training set subjects into a first group of high responders to the known therapy, and a second group of low responders to the known therapy, wherein the first group of high responders is characterized by survival time longer than average survival time for the entire training set of subjects, the second group of low responders is characterized by survival time shorter than average survival time for the entire training set of subjects.

3. The method as in claim 2, wherein the mathematical algorithm is further trained to define a first subset of RNA-based biomarkers corresponding to dividing all training set subjects into the first group of high responders and the second group of low responders.

4. The method as in claim 3, wherein a presence of a TP53 mutation is a predictor for a second group of low responders.

5. The method as in claim 2, wherein the mathematical algorithm is further trained to subdivide the first group of high responders into a third group of high responders and a fourth group of high responders, wherein the third group of high responders is characterized by survival time longer than average survival time for the entire first group of high responders, the fourth group of high responders is characterized by survival time shorter than average survival time for the entire first group of high responders.

6. The method as in claim 5, wherein the mathematical algorithm is further trained to define a second subset of RNA-based biomarkers corresponding to dividing all subjects of the first group of high responders into the third group of high responders and the fourth group of high responders.

7. The method as in claim 6, wherein the second subset of RNA-based biomarkers is different from the first subset of RNA-based biomarkers.

8. The method as in claim 7, wherein the mathematical algorithm is further trained to subdivide the second group of low responders into a fifth group of low responders and a sixth group of low responders, wherein the fifth group of low responders is characterized by survival time longer than average survival time for the entire second group of low responders, the sixth group of low responders is characterized by survival time shorter than average survival time for the entire second group of low responders.

9. The method as in claim 8, wherein the mathematical algorithm is further trained to define a third subset of RNA-based biomarkers corresponding to dividing all subjects of the second group of low responders into the fifth group of low responders and the sixth group of low responders.

10. The method as in claim 9, wherein the third subset of RNA-based biomarkers is different from the first subset of RNA-based biomarkers.

11. The method as in claim 2, wherein treating the subject in step (d) comprises:
a step of treating the subject forecasted in step (c) as a high responder with the known therapy;
a step of treating the subject forecasted in step (c) as a low responder with a further therapy or an additional therapy; or
a combination thereof.

12. The method as in claim 1, wherein the mathematical algorithm is based on a naïve Bayesian classifier that is a generalized naïve Bayesian classifier defined by applying a geometric mean to a likelihood product.

13. The method as in claim 12, wherein the naïve Bayesian classifier is trained to rank individual RNA-based biomarkers from initial set of available RNA-based biomarkers that includes at least 500 individual genes.

14. The method as in claim 13, wherein at least some of the individual RNA-based biomarkers are cross-validated by subdividing the training set of subjects into a plurality of subsets, constructing a naïve Bayesian classifier for the individual RNA-based biomarker for one of the subsets and verifying the same RNA-based biomarker for at least some of the remaining subsets thereby reducing noise and overfitting.

15. The method as in claim 14, wherein:
after cross-validation the number of ranked RNA-based biomarkers is between 50 and 70 for each of the subdividing step of the first group and the second group, the third group and the fourth group, and the fifth group and the sixth group of the training set of subjects;
the set of individual RNA-based biomarkers for dividing the entire training set of subjects into the first group and the second group is different from the respective set of individual RNA-based biomarkers for subdividing the first group of high responders into the third group and the fourth group; and
the set of individual RNA-based biomarkers for dividing the entire training set of subjects into the first group and the second group is different from the respective set of individual RNA-based biomarkers for subdividing the second group of low responders into the fifth group and the sixth group.

16. The method as in claim 15, wherein:
the set of RNA-based biomarkers for dividing the training set into the first group and the second group is selected from a group consisting of PPP2R1B, GOLGA5, LINGO2, HMGA1, SIN3A, ARID1A, BCL7A, CDK5RAP2, MAGED1, CREB3L1, AMER1, DLL1, GSTT1, GPR34, DNM2, CCNB1IP1, MUTYH, RET, CDH1, POFUT1, XRCC6, KIT, RALGDS, SS18, CD22, BRCA2, HDAC3, LHX4, FAM19A2, PRG2, PRCC, TBL1XR1, HIF1A, EDIL3, ROS1, DKK4, CDC25A, WNT7B, MYBL1, MLLT10, SLCO1B3, TACC2, CANT1, NCAM1, FGF3, FGF19, PPP3R2, CRADD, ETV6, SPP1, SDHB, FGF2, SUZ12, MB21D2, MYC, BAX, CEP57, ITGA5, ABCC3, and HECW1;
the set of RNA-based biomarkers for dividing the first group of the training set into the third group of high responders and the fourth group of high responders is selected from a group consisting of DUSP22, CTNNA1, DUX2, SSX1, SSX2, CTNNB1, DCLK2, FH, DUSP9, FCGR2B, STAT5B, ESR1, CD274, TERF1, AKAP9, DGKI, HMGA1, ARNT, MAFB, PPP3CC, COL3A1, NUTM2A, CIT, MGMT, CDK6, SORT1, RCSD1, CDK5RAP2, SIN3A, RABEP1, MB21D2, KDR, SS18L1, SSBP2, SH2D5, ASXL1, AMER1, AFF1, PRKCD, 2-Sep, TPM4, FIGF, NODAL, GRM3, STAT6, GAB1, RPL22, BDNF, SNX29, MELK, ARRDC4, FGF10, MMP9, YY1AP1, HAS2, DLEC1, DEK, TLL2, BCL2L2, and ID3;
the set of RNA-based biomarkers for dividing the second group of low responders of the training set into the fifth group and the sixth group is selected from a group consisting of AHI1, EPHA5, DUSP22, DUSP26, DUSP9, DUX2, MGMT, MIB1, MIPOL1, MIR1260B, MIR4321, MIR4683, MIR4758, MIR6515, MIR6752, MIR6765, BIVM-ERCC5, SSX1, SSX2, LTBP1, MAFB, TLR4, CTNNB1, ETV5, CHEK2, FUS, SS18L1, SSBP2, DGKI, CIT, TFE3, FGF19, TRIM33, CTCF, LAMA1, TBL1XR1, TOP1, RB1, OLR1, DOCK1, ARID1A, RABEP1, EP400, STK11, ETS1, MAPK1, CDC14A, LMO7, SS18, ICK, FLI1, POU5F1, RCSD1, HRAS, BACH2, CDK7, GAS5, CARS, SRSF2, and MAP3K6; or
combinations thereof.

17. A method for identifying one or more individual RNA-based biomarkers for forecasting clinical course of a subject with a heterogeneous disease, wherein the heterogeneous disease is defined as a group of biologically diverse conditions affecting same cells or tissues and causing same or similar symptoms, the method comprising the following steps:
a. providing a training set of subjects with the heterogenous disease with known plurality of individual RNA-based biomarkers and known survival time;
b. based on survival time, dividing all subjects from the training set into a first group of high responders and a second group of low responders, and
c. using machine learning, identifying a first subset of one or more individual RNA-based biomarkers from a plurality of individual RNA-based biomarkers, wherein the first subset of one or more individual RNA-based biomarkers is identified as correlating to dividing the subjects into the first group and the second group.

18. The method as in claim 17 further comprising a step (d) of dividing the first group of high responders into a third group of high responders and a fourth group of high responders, wherein the third group of high responders is characterized by survival time longer than average survival time for the entire first group of high responders, the fourth group of high responders is characterized by survival time shorter than average survival time for the entire first group of high responders.

19. A method for treating a subject with diffuse large B-cell lymphoma, comprising a step of using a Bayesian classifier to define the subject as a high responder or a low responder to chemotherapy using one or more of individual RNA-based biomarkers selected from a group consisting of PPP2R1B, GOLGA5, LINGO2, HMGA1, SIN3A, ARID1A, BCL7A, CDK5RAP2, MAGED1, CREB3L1, AMER1, DLL1, GSTT1, GPR34, DNM2, CCNB1IP1, MUTYH, RET, CDH1, POFUT1, XRCC6, KIT, RALGDS, SS18, CD22, BRCA2, HDAC3, LHX4, FAM19A2, PRG2, PRCC, TBL1XR1, HIF1A, EDIL3, ROS1, DKK4, CDC25A, WNT7B, MYBL1, MLLT10, SLCO1B3, TACC2, CANT1, NCAM1, FGF3, FGF19, PPP3R2, CRADD, ETV6, SPP1, SDHB, FGF2, SUZ12, MB21D2, MYC, BAX, CEP57, ITGA5, ABCC3, and HECW1.

20. The method as in claim 76, wherein treating the subject in step (d) comprises:
a step of treating the subject forecasted as a high responder with the known therapy;
a step of treating the subject forecasted as a low responder with a further therapy or an additional therapy; or
a combination thereof.
